(19) United States
(12) Patent Application Publication
(10) Pub. No.: US 2011/0218404 A1
(43) Pub. Date: Sep. 8, 2011

(54) LIGHT SENSING SYSTEM AND ENDOSCOPE SYSTEM

(75) Inventor: Katsumi HIRAKAWA, Sagamihara-shi (JP)

(73) Assignee: OLYMPUS MEDICAL SYSTEMS CORP., Tokyo (JP)

(21) Appl. No.: 12/898,972

(22) Filed: Oct. 6, 2010

Related U.S. Application Data
(63) Continuation of application No. PCT/JP2010/057454, filed on Apr. 27, 2010.

(30) Foreign Application Priority Data
Jun. 3, 2009 (JP) 2009-134325

(51) Int. Cl. A61B 1/06; G01N 21/00

(52) U.S. Cl. 600/178; 356/73.1

(57) ABSTRACT

The medical instrument includes an optical fiber sensor in which a plurality of FBG sections are formed, a reflector that forms reference light, a light source that outputs light by stepwise changing light beams of wavelengths of a predetermined interval, a coupler that splits light and generates interference light, a detection section that detects the interference light and a calculation section that calculates amounts of deformation of the FBG sections based on the detection result of the detection section.

### CROSS REFERENCE TO RELATED APPLICATION

This application is a continuation application of PCT/JP2010/057454 filed on Apr. 27, 2010 and claims benefit of Japanese Application No. 2009-134325 filed in Japan on Jun. 3, 2009, the entire contents of which are incorporated herein by this reference.

### BACKGROUND OF THE INVENTION

1. Field of the Invention

The present invention relates to a light sensing system having an optical fiber sensor in which a fiber Bragg grating sensor section is created and an endoscope system provided with the light sensing system, and more particularly, to a light sensing system using an optical frequency domain reflectometry multiplexing scheme and an endoscope system provided with the light sensing system.

2. Description of the Related Art

A fiber Bragg grating (hereinafter referred to as “FBG”) sensor is a sensor with a grating section of a varying refractive index created in a core section of an optical fiber and its grating section reflects light of a predetermined wavelength of incident light. This predetermined wavelength is called “Bragg wavelength.” In the FBG sensor, when the grating section expands or contracts in its longitudinal direction, the Bragg wavelength changes. For this reason, the FBG sensor is used for temperature measurement, distortion measurement or the like.

When an optical frequency domain reflectometry multiplexing (hereinafter referred to as “OFDR”) scheme is applied to an optical fiber sensor, a plurality of FBG sensor sections of the same Bragg wavelength are formed into a single optical fiber. Reflected light from a reflector which is a total reflection termination is used as reference light and caused to interfere with reflected light from the optical fiber sensor to thereby detect the degree to which the respective FBG sensor sections have deformed, in other words, the degree of distortion that has occurred. Fiber sensors using an OFDR scheme are used as distortion measuring sensors for aircraft, buildings or the like.

For example, Japanese Patent Application Laid-Open Publication No. 2003-515104 and Japanese Patent Application Laid-Open Publication No. 2004-251779 disclose a shape measuring apparatus using an optical fiber sensor that measures three-dimensional shapes. In the case of a shape measuring apparatus that measures three-dimensional shapes, measuring three-dimensional deformations of respective measuring locations requires at least three FBG sensor sections to be arranged at the respective measuring locations and three or more optical fiber sensors are used.

Compared to a light sensing system that uses an optical fiber sensor having FBG sensor sections of different Bragg wavelengths, an OFDR-based light sensing system can perform measurement even if more FBG sensor sections are formed into a single optical fiber no matter how large distortion of a detection target may be. Thus, the OFDR-based light sensing system can perform sensing using fewer optical fiber sensors and is suitable for use in a system requiring diameter reduction.

### SUMMARY OF THE INVENTION

The light sensing system of the present invention includes an optical fiber sensor in which a plurality of fiber Bragg grating sensor sections are formed, a light source that outputs light by stepwise changing frequencies with a passage of time at a frequency interval of ½ times or less a full width at half maximum of a reflected light spectrum determined by characteristics of the fiber Bragg grating sensor sections, a light supply section that supplies the light outputted from the light source to the optical fiber sensor, a reference light forming section that forms reference light to be caused to interfere with reflected light from the optical fiber sensor, an interference section that generates interference light by causing the reference light to interfere with the reflected light, a detection section that detects the interference light from the interference section and a calculation section that calculates amounts of deformation of the plurality of fiber Bragg grating sensor sections based on the detection result of the detection section.

The endoscope system of the present invention is provided with a light sensing system, including an optical fiber sensor disposed in an insertion portion of an endoscope in which a plurality of fiber Bragg grating sensor sections are formed, a light source that outputs light by stepwise changing frequencies with a passage of time at a wavelength interval of ½ times or less a full width at half maximum of a reflected light spectrum determined by characteristics of the fiber Bragg grating sensor sections, a light supply section that supplies the light outputted from the light source to the optical fiber sensor, a reference light forming section that forms reference light to be caused to interfere with reflected light from the optical fiber sensor, an interference section that generates interference light by causing the reference light to interfere with the reflected light, a detection section that detects the interference light from the interference section and a calculation section that calculates amounts of deformation of the plurality of fiber Bragg grating sensor sections and the shape of the insertion portion based on the detection result of the detection section.

### BRIEF DESCRIPTION OF THE DRAWINGS

FIG. 1 is a perspective schematic cross-sectional view illustrating a configuration of an optical fiber sensor;

FIG. 2 is a configuration diagram illustrating an OFDR-based light sensing system;

FIG. 3A is a diagram illustrating a signal of light in the OFDR-based light sensing system;

FIG. 3B is a diagram illustrating a signal of light in the OFDR-based light sensing system;

FIG. 3C is a diagram illustrating a signal of light in the OFDR-based light sensing system;

FIG. 4 is a diagram illustrating a situation in which a medical instrument according to a first embodiment is used;

FIG. 5 is a diagram illustrating the medical instrument of the first embodiment;

FIG. 6A is a cross-sectional configuration diagram in a longitudinal direction illustrating a configuration of the optical fiber sensor of the medical instrument of the first embodiment;

FIG. 6B is a cross-sectional view along a line VIB-VIB of FIG. 6A illustrating a configuration of the optical fiber sensor of the medical instrument of the first embodiment;

FIG. 7 is a configuration diagram of the medical instrument of the first embodiment;

FIG. 8A is a diagram illustrating signal processing in the medical instrument of the first embodiment;

FIG. 8B is a diagram illustrating signal processing in the medical instrument of the first embodiment;

FIG. 8C is a diagram illustrating signal processing in the medical instrument of the first embodiment;

FIG. 9 is a diagram illustrating a time variation of a frequency of light outputted from a continuous wavelength sweep laser;

FIG. 10 is a diagram illustrating a time variation of a wavelength of light outputted from a discrete wavelength sweep laser;

FIG. 11A is a diagram illustrating a signal of light in an OFDR-based light sensing system using a continuous wavelength sweep laser;

FIG. 11B is a diagram illustrating a signal of light in the OFDR-based light sensing system using a continuous wavelength sweep laser;

FIG. 11C is a diagram illustrating a signal of light in the OFDR-based light sensing system using a continuous wavelength sweep laser;

FIG. 11D is a diagram illustrating a signal of light in the OFDR-based light sensing system using a continuous wavelength sweep laser;

FIG. 12A is a diagram illustrating a signal of light in the OFDR-based light sensing system using a discrete wavelength sweep laser;

FIG. 12B is a diagram illustrating a signal of light in the OFDR-based light sensing system using a discrete wavelength sweep laser;

FIG. 12C is a diagram illustrating a signal of light in the OFDR-based light sensing system using a discrete wavelength sweep laser;

FIG. 12D is a diagram illustrating a signal of light in the OFDR-based light sensing system using a discrete wavelength sweep laser;

FIG. 13 is a diagram illustrating a relationship between a variation in central light wavelength and time and a relationship between a variation in central optical frequency and wavelength resolution;

FIG. 14A is a diagram illustrating a signal of light in an OFDR-based light sensing system using a discrete wavelength sweep laser;

FIG. 14B is a diagram illustrating a signal of light in the OFDR-based light sensing system using a discrete wavelength sweep laser;

FIG. 15 is a diagram illustrating a signal of light in the OFDR-based light sensing system using a discrete wavelength sweep laser;

FIG. 16 is a diagram illustrating a signal of light in the OFDR-based light sensing system using a discrete wavelength sweep laser;

FIG. 17 is a diagram illustrating measurement accuracy of a light sensing system; and

FIG. 18 is a configuration diagram of a medical instrument according to a second embodiment.

### DETAILED DESCRIPTION OF THE PREFERRED EMBODIMENTS

### <About FBG Sensor>

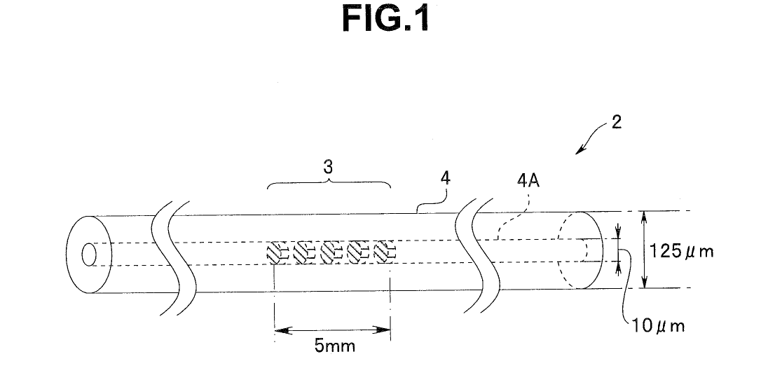

First, an FBG sensor will be described in brief. As shown in FIG. 1, an FBG sensor section 3 is a diffraction grating in which a refractive index of a core section 4A having a diameter of 10 μm periodically varies over a predetermined length (5 mm) of an optical fiber 4 having a diameter of 125 μm. The refractive index is slightly increased through a photorefractive effect by irradiating the germanium-doped core section 4A with ultraviolet rays via a mask. Using this, the FBG sensor section 3 is made by periodically forming portions of a high refractive index (grating) in the axial direction. The number of gratings and the grating width with respect to the axial direction of the core section in FIG. 1 or the like are different from the actual FBG sensor section for ease of understanding of the structure.

Of the incident light, the FBG sensor section 3 reflects only light of a Bragg wavelength λB which is a predetermined wavelength expressed by the following equation according to an interval d of the diffraction grating, namely, a period.

λB=2×n×d

where n is a refractive index of the core section 4A.

For example, when the refractive index n of the core section 4A is 1.45 and the Bragg wavelength λB is 1550 nm, the interval d of the diffraction grating is on the order of 0.53 μm.

As is apparent from the above described equation, when the FBG sensor section 3 expands, the interval d of the diffraction grating also increases, and therefore the Bragg wavelength λB moves toward the long wavelength side. On the contrary, when the FBG sensor section 3 contracts, the interval d of the diffraction grating is also reduced, and therefore the Bragg wavelength λB moves toward the short wavelength side. For this reason, the FBG sensor section 3 can be used as a sensor for detecting a temperature or an amount of distortion or the like.

According to the specification of the FBG sensor section 3, the reflected light of the FBG sensor section 3 has a predetermined bandwidth. Suppose the full width at half maximum of the reflected light spectrum is Δλ_{FBG}. The waveform of the reflected light in the time domain is, for example, a Gaussian distribution shape.

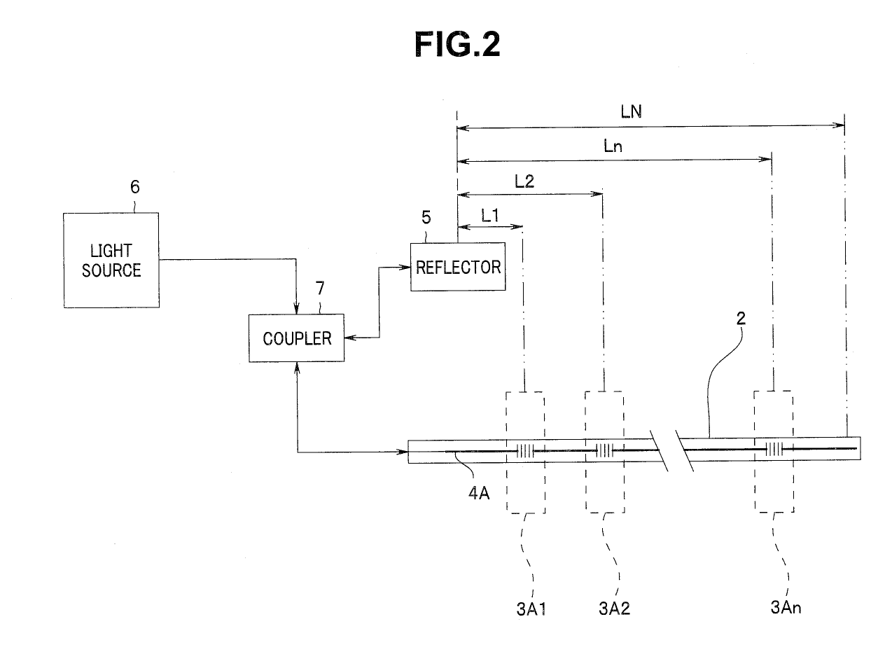

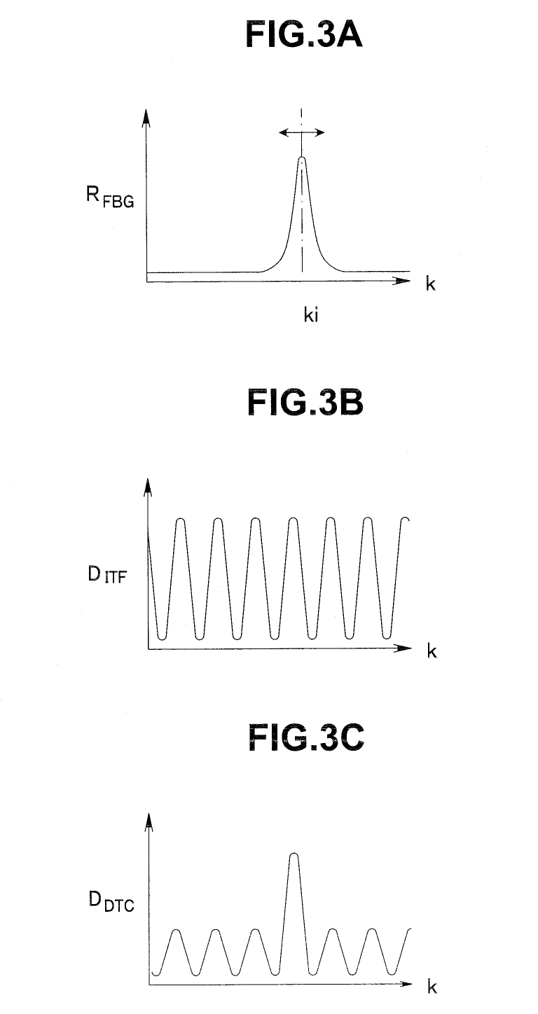

Next, the principle of detection of the optical fiber sensor 2 according to an OFDR scheme will be described using FIG. 2 and FIG. 3A to FIG. 3C. As shown in FIG. 2, light emitted from a light source 6 is split by a coupler 7 and supplied to an optical fiber sensor 2 and a reflector 5. The reflector 5 is a total reflection termination as reference light forming means for forming reference light to be caused to interfere with the reflected light from the optical fiber sensor 2 and the coupler 7 is not only light supply means but also interference means for causing reflected light reflected by the FBG sensor section 3 of the optical fiber sensor 2 to interfere with the reference light.

The optical fiber sensor 2 has n FBG sensor sections 3A1 to 3An and suppose the difference between the distance from the light source 6 to the FBG sensor sections 3A1, 3A2, . . . , 3An and the distance from the light source 6 to the reflector 5 is L1, L2, . . . , Ln. LN is the difference between the distance from the light source 6 to the termination of the optical fiber sensor 2A and the distance from the light source 6 to the reflector 5. In the optical fiber sensor 2, the interval difference between the distance of each of the n FBG sensor sections 3 to the coupler 7 and the distance from the coupler 7 to the reflector 5 differs from each other.

As has already been described, the FBG sensor section 3 strongly reflects only light of a Bragg wavelength λB which is a specific wavelength, the relationship between a light wave number k of the light source 6 and its reflected light strength R_{FBG} is as shown in FIG. 3A. Furthermore, the light wave number k indicating a peak varies depending on the magnitude of distortion of the FBG sensor section 3.

The wavelength (λ) of light, frequency (f) of light and light wave number (k) are parameters showing attributes of light. That is, k=2π/λ, λ=c/f (c: velocity of light).

The reflected light from the FBG sensor section 3 and the reference light which is reflected light from the reflector 5 have an optical path difference 2π Li (i=1, 2, . . . , n). Two reflected light beams having an optical path difference produce interference and a fluctuation component of the interference light intensity except a DC component has a shape as shown in FIG. 3B depending on the light wave number k and is expressed as follows.

D_{ITF}=A cos(2nLik)

where n denotes a refractive index of the optical fiber. Through the aforementioned operation, the intensity D_{DTC} of the interference light varies in a shape having a certain period and a peak with respect to the light wave number k as shown in FIG. 3C. That is, the intensity D_{DTC} is expressed by the following equation.

D_{DTC}=R_{FBG}(k)cos(2nLik)

Here, R_{FBG}(k) is a function of light wave number (wavelength) expressing reflection characteristics of the FBG sensor section 3. It is possible to measure an optical path difference Li (i=1, . . . , n), that is, the position of the FBG sensor section 3 from the period of the interference signal and measure the amount of deformation of the FBG sensor section 3 from the light wave number k showing a peak. As will be described later, the position and the amount of deformation of the FBG sensor section 3 are actually calculated from the frequency difference by analyzing the frequency of the interference signal and comparing the result of frequency analysis with a result of frequency analysis when no deformation occurs. In the FBG sensor section 3 as a whole, the light intensity is observed as the sum of optical path differences Li (i=1, . . . , n), that is, waveforms having different periods. Here, although one entire FBG has been described as one sensor, the OFDR scheme focuses on one of a plurality of FBG sensor sections 3Ai (i=1, . . . , n) and can analyze the amount of distortion and the position of distortion produced with position accuracy of 1 mm or less.

### First Embodiment

Hereinafter, a medical instrument 1 which is a light sensing system according to a first embodiment of the present invention will be described with reference to the accompanying drawings.

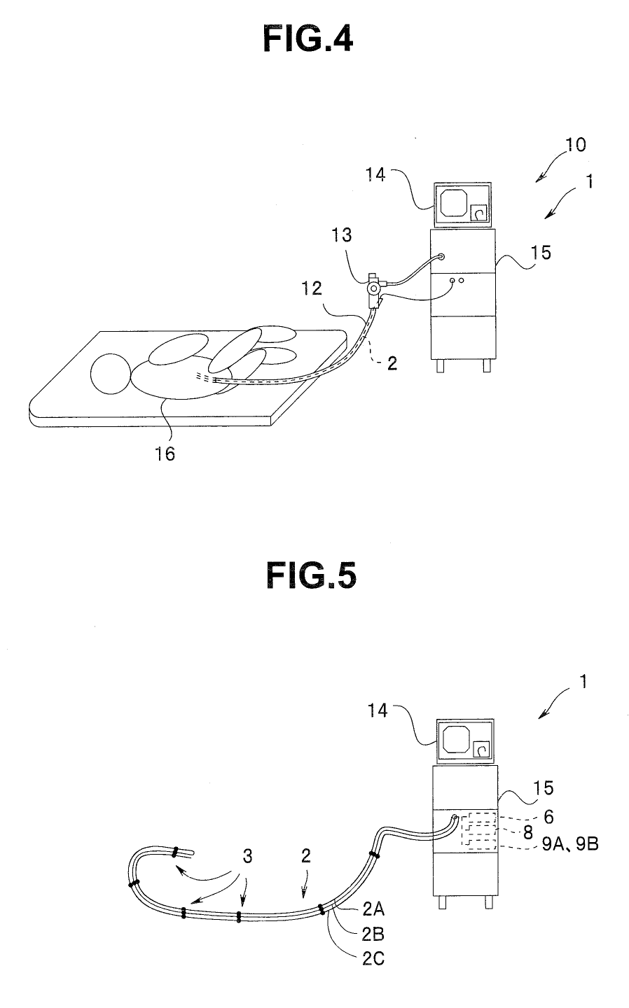

As shown in FIG. 4 and FIG. 5, the medical instrument 1 which is the light sensing system of the first embodiment can measure the shape of an insertion portion 12 of an endoscope of an endoscope system 10. The endoscope system 10 includes the elongated insertion portion 12 which is a medical instrument inserted into the body of a subject 16 for conducting observation or treatment, an operation portion 13 for operating the insertion portion 12, a main unit 15 that performs control and image processing or the like on the entire endoscope system 10 and a monitor 14 that displays an endoscope image or the like. An optical fiber sensor 2 of the medical instrument 1 is inserted into a channel 12A (not shown) from a treatment instrument hole which is an opening on the operation portion 13 side of the channel that passes through the insertion portion 12 and disposed so as to deform into the same shape as the insertion portion 12. The monitor 14 of the endoscope system 10 also functions as display means of the medical instrument 1 and can display the shape of the optical fiber sensor 2, that is, the shape of the insertion portion 12 on the same screen as the endoscope image. The optical fiber sensor 2 may be built in the insertion portion 12 instead of being inserted into the channel 12A.

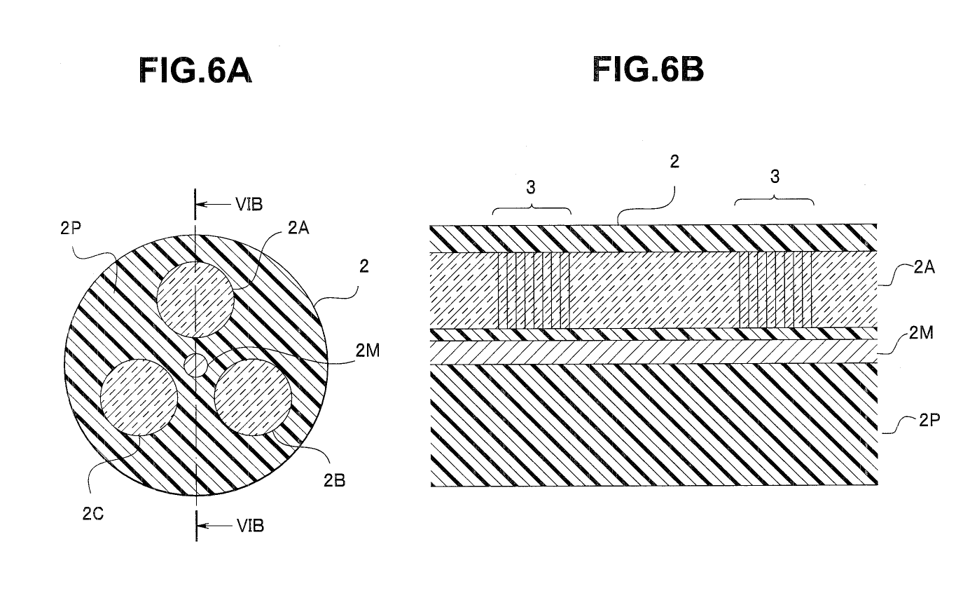

As shown in FIG. 6A and FIG. 6B, the optical fiber sensor 2 is a fiber array made up of three optical fiber sensors 2A, 2B and 2C bundled together around a metal wire 2M via resin 2P and has flexibility. As shown in FIG. 2, the optical fiber sensors 2A, 2B and 2C are provided with their respective FBG sensor sections 3 at the same position in the axial direction. That is, since the three FBG sensor sections 3 are located at the same position, in the optical fiber sensor 2 displacement in a three-dimensional space of a portion of the insertion portion 12 where the three FBG sensor sections 3 are arranged can be measured.

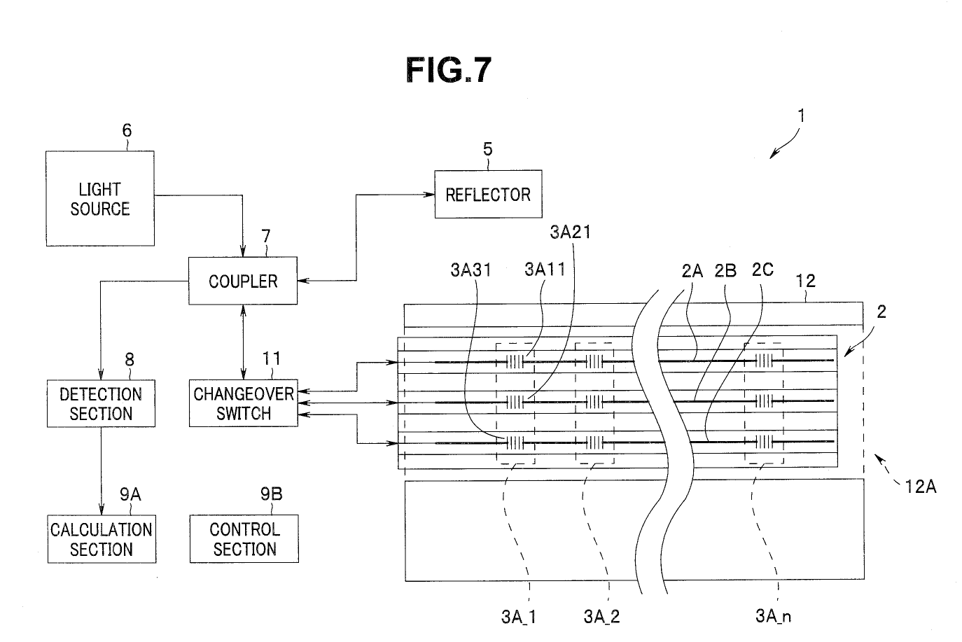

As shown in FIG. 7, the medical instrument 1 includes the optical fiber sensor 2, a light source 6 that time-sequentially and stepwise changes and outputs light beams of wavelengths of a predetermined interval arranged in the main unit 15, and a coupler 7, an optical part, that is light splitting means for splitting light emitted from the light source 6 to be supplied to the optical fiber sensor 2 and the reflector 5 which is reflection means and also interference means for causing light reflected from the reflector 5 and light reflected from the FBG sensor section 3 of the optical fiber sensor 2 to interfere with each other. That is, the light splitting means and the interference means are configured with the coupler 7 which is a single optical part. Of course, the light splitting means and the interference means may be configured with different members. A changeover switch 11 is disposed between the coupler 7 and the optical fiber sensor 2 and light is sequentially supplied to three optical fiber sensors 2A, 2B and 2C. The changeover switch 11 changes an optical path in synchronization with the timing of wavelength sweep of the light source 6 under the control of a control section 9B. In other words, the control section 9B controls the changeover switch 11 so that light is supplied to a different optical fiber sensor 2 every time the light source 6 performs wavelength sweep once.

As the light source 6, for example, a super structure grating distributed Bragg reflector laser (SSG-DBR laser) which is a wideband wavelength variable laser light source may be used. To be more specific, a light source 6 with 400 channels may be used, which stepwise changes and outputs laser beams, for example, in a band of 1533.17 to 1574.13 nm in 0.1 nm wavelength steps (intervals):λs and at a channel step speed of 10 μs/step. Since wideband wavelength variable lasers, which are discrete wavelength sweep lasers, are mass-produced for communication use, these are cheaper than continuous wavelength sweep lasers which are used for special purposes, and are available at 1/10 price.

The medical instrument 1 is further provided with a detection section 8 which is detection means for detecting interference light from the coupler 7 by converting it to an electric signal, a calculation section 9A which is calculating means for calculating an amount of wavelength shift (difference between the wavelength when there is no deformation in the portion where the FBG sensor section 3 exists and the wavelength when there is deformation) of each FBG sensor section 3 using a digital signal generated through AD conversion from the signal detected by the detection section 8, determining the amount of deformation of the FBG sensor section 3 from the calculated amount of wavelength shift and calculating the shape of the optical fiber sensor 2 from the amount of deformation of each FBG sensor section 3 and the control section 9B that controls the entire medical instrument 1.

Next, a detection method according to an OFDR scheme will be described in further detail taking a case where light is supplied to the optical fiber sensor 2A by the changeover switch 11 in the medical instrument 1 as an example. As shown in FIG. 7, the light emitted from the light source 6 is branched by the coupler 7. One portion of the branched light is reflected by the reflector 5 and returned to the coupler 7 again. The other portion of the branched light is reflected by the FBG sensor section 3 of the optical fiber sensor 2A via the changeover switch 11 and is returned to the coupler 7 again. The reflected light from the reflector 5 (hereinafter also referred to as “reflected light of laser”) and the reflected light from the FBG sensor section 3 (hereinafter also referred to as “reflected light of FBG”) form interference light at the coupler 7 which also serves as the interference means and the interference light is measured as an interference signal at the detection section 8. The detection section 8 is a light receiver and measures the interference signal.

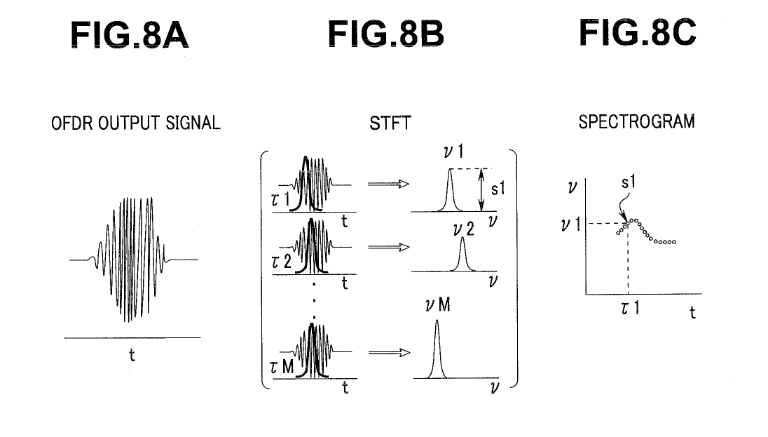

The calculation section 9A then applies short-time Fourier transform (hereinafter referred to as “STFT”) processing to the interference signal and thereby obtains three-dimensional information made up of distance information, distortion information and reflection intensity information. That is, as shown in FIG. 8A to FIG. 8C, part of the interference signal is extracted by focusing on a signal in a time zone of a time window width (Δτ) of a time-varying interference signal (FIG. 8A) and multiplying the interference signal by the time window (FIG. 8B). Information is extracted by applying STFT processing to the extracted part of the interference signal. For example, FIG. 8C is an example where the extracted three-dimensional information is displayed on a two-dimensional plane, and displays reflected light intensity s1 in color tone with the horizontal axis showing time t and the vertical axis showing STFT frequency ν. Since the light from the light source 6 is subjected to wavelength sweep, time t on the horizontal axis in FIG. 8C corresponds to the wavelength λ of light. Since the wavelength λ of the interference signal decreases as the optical path difference increases, the STFT frequency ν on the vertical axis corresponds to the distance.

Next, the wavelength resolution (Δλ) and distance resolution (ΔL) in the medical instrument 1 of the present embodiment having an SSG-DBR laser which is a discrete wavelength sweep laser as the light source 6 will be described in comparison with a case using a continuous wavelength sweep laser as a light source.

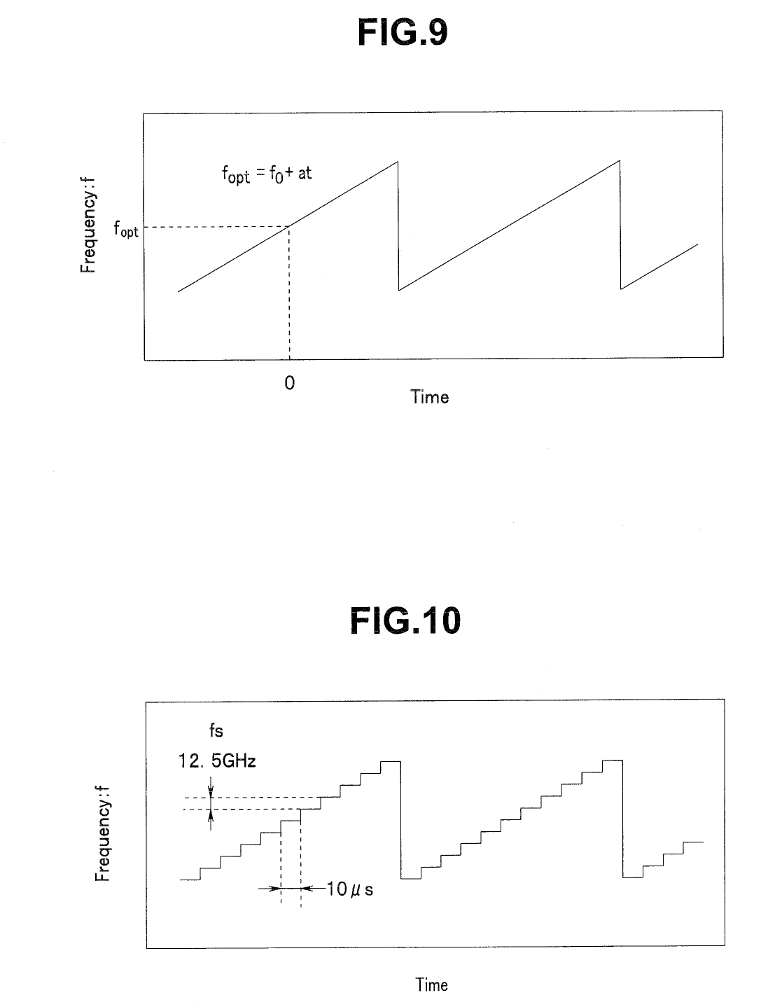

FIG. 9 illustrates a time variation of frequency of light outputted from the continuous wavelength sweep laser that can continuously change the wavelength of light outputted and FIG. 10 illustrates a time variation of wavelength of light outputted from the SSG-DBR laser that stepwise changes and outputs light beams of wavelengths of a predetermined interval (λs).

As shown in FIG. 9, the laser output intensity of the continuous wavelength sweep laser can be expressed by the following equation.

fₒₚₜ=f₀+at

where fₒₚₜ denotes a frequency of light outputted from the continuous wavelength sweep laser and f₀ denotes a frequency at time 0 and a denotes a constant of proportion.

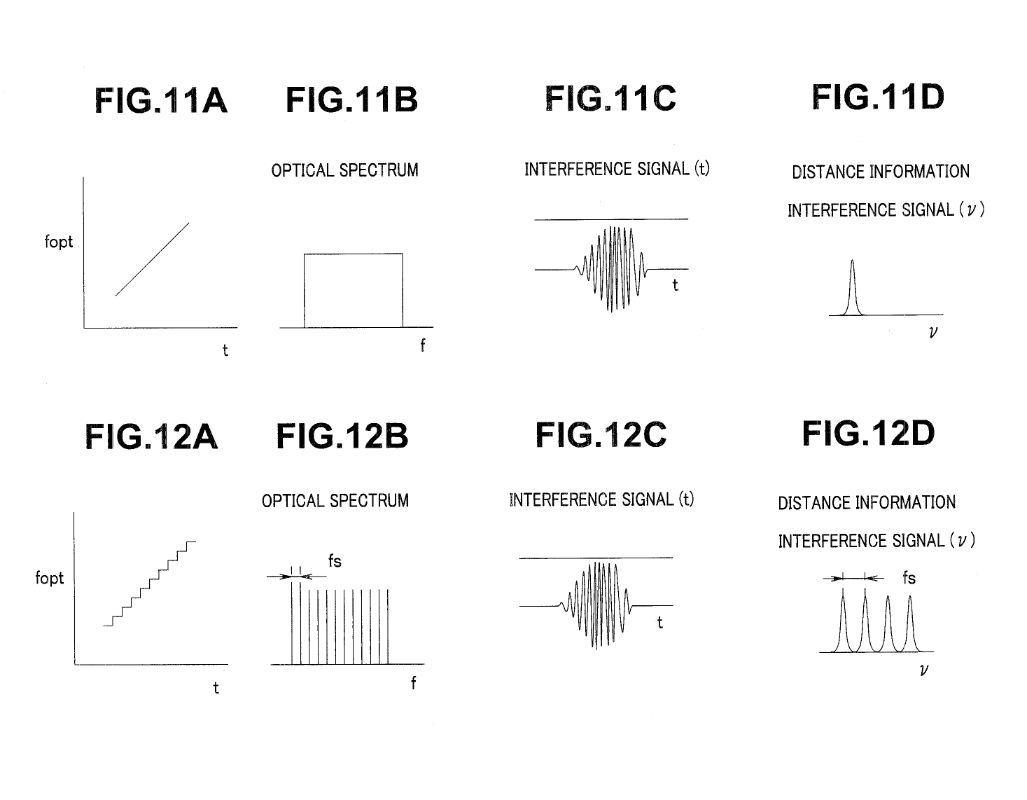

Next, FIG. 11A illustrates a frequency variation of light for one period outputted from the continuous wavelength sweep laser shown in FIG. 9. As shown in FIG. 11B, an optical spectrum of the light outputted from the continuous wavelength sweep laser is rectangular. For this reason, an interference signal (ν) resulting from applying Fourier transform processing to the interference signal (t) shown in FIG. 11C is a single sinc function shown in FIG. 11D as has already been described. ν on the horizontal axis at the peak position of the interference signal (ν) in FIG. 11D shows a frequency, that is, distance information and the intensity on the vertical axis shows reflected light intensity.

By contrast, FIG. 12A shows a frequency variation for one period of light outputted from the discrete wavelength sweep laser shown in FIG. 10. As shown in FIG. 12B, the optical spectrum of light outputted from the discrete wavelength sweep laser is comb-shaped having many peaks. For this reason, the interference signal (ν) resulting from applying Fourier transform processing to the interference signal (t) shown in FIG. 12C is a plurality of sinc functions located at an interval of (1/λs) as shown in FIG. 12D. For this reason, in the medical instrument 1 of the present embodiment having a discrete wavelength sweep laser as the light source 6, measurable lengths (measurement range) are restricted. This is the same problem as aliasing of discrete Fourier transform. That is, measurement needs to be performed within a measurement distance range in which especially fundamental waves of a sinc function do not overlap with each other.

Hereinafter, conditions for the calculation section 9A of the medical instrument 1 to calculate position information and wavelength information, that is, position and amount of deformation of each FBG sensor section 3 will be studied.

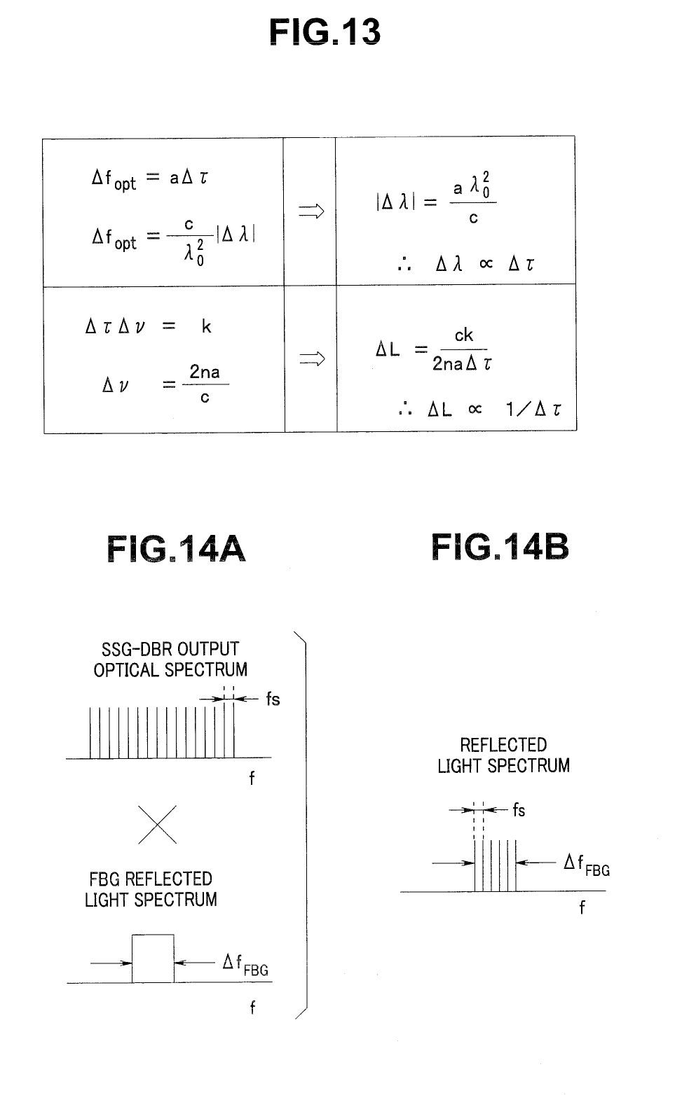

First, the time window width (Δτ), wavelength resolution (Δλ) and distance resolution (ΔL) in STFT processing will be described. As shown in FIG. 13, from the relationship between a variation Δfopt of the center optical frequency (fopt) and time, and the relationship between a variation Δfopt of the center optical frequency (fopt) and wavelength resolution (Δλ) the wavelength resolution wavelength resolution (Δλ) is proportional to the time window width time window width (Δτ). Furthermore, from the uncertainty principle of Fourier transform and the relationship between a frequency variation and distance resolution (ΔL) of the interference signal, the distance resolution (ΔL) is inversely proportional to the time window width (Δτ). That is, it is understandable that the distance resolution (ΔL) and the wavelength resolution (Δλ) have a trade-off relationship that pursuing one cannot help but sacrifice the other.

Here, consider a wavelength spectrum from the optical fiber sensor 2 in the case of a discrete wavelength sweep laser. As shown in FIG. 14A, the result of multiplying the output optical spectrum of the SSG-DBR laser by the FBG reflection spectrum finally becomes a reflected light spectrum from the optical fiber sensor 2 shown in FIG. 14B. In FIG. 14A and FIG. 14B, the wavelength interval of light changed and outputted stepwise by the discrete wavelength sweep laser is λs and fs is a step frequency calculated from c/λs. Here, c is the velocity of light in vacuum.

Furthermore, Δf_{FBG} is a parameter full width at half maximum indicating the expansion of a peak of reflected light from each FBG sensor section 3, and is simply displayed as rectangular.

In the medical instrument 1 having the discrete wavelength sweep laser, when a relationship of (Δf_{FBG}≧2fs) holds, the calculation section 9A can calculate the positions and wavelength information of the respective FBG sensor sections 3.

For example, as shown in FIG. 14B, when the relationship of (Δf_{FBG}≧2fs) holds, this means that there are three or more peaks of the spectrum of output light (reflected light) of the SSG-DBR laser in the spectrum (Δf_{FBG}) of the reflected light of FBG.

In other words, when the relationship of (Δf_{FBG}≧2fs) does not hold, the wavelength resolution (Δλ) is determined by fs. The condition for calculating at least position information is (fs≦0.5×Δf_{FBG}). However, the calculation section 9A cannot always calculate the shape of the insertion portion 12 with desired accuracy (resolution) according to the above described condition alone.

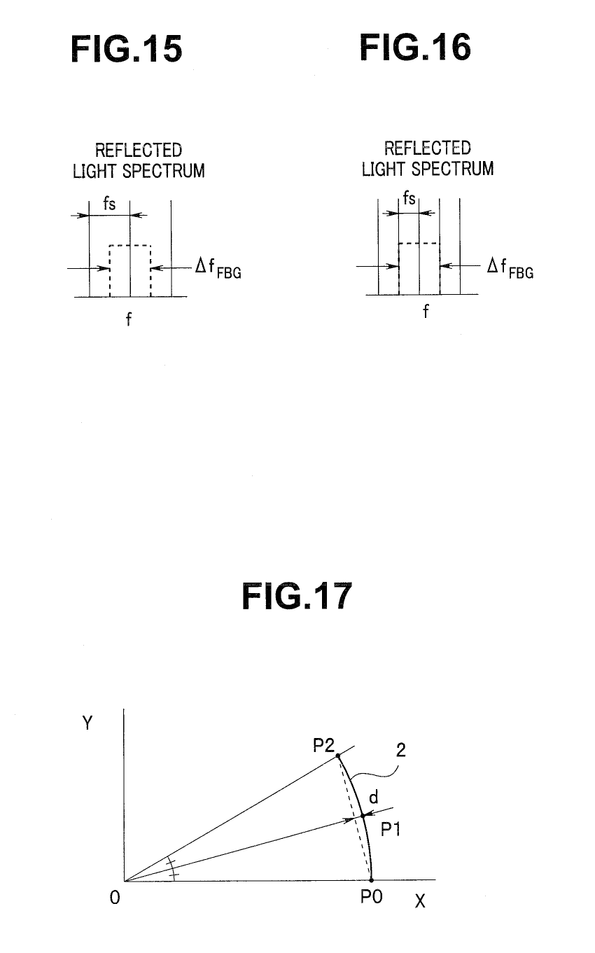

Furthermore, when (Δf_{FBG}<2fs), the calculation section 9A can calculate neither the position information nor the wavelength information. When only one laser spectrum exists in the reflected light spectrum as shown in, for example, FIG. 15, only the same optical spectrum as that of the laser of continuous light is calculated and the interference signal becomes DC. This is because an impulse waveform is subjected to Fourier transform. That is, when the impulse waveform is defined to be 1 only when t=t0 and 0 otherwise, the Fourier transform result of the impulse waveform becomes 1 at all frequencies.

By contrast, when (Δf_{FBG}≧2fs), the calculation section 9A can calculate the position information and wavelength information. FIG. 16 illustrates a reflected light spectrum when (Δf_{FBG}≈>2fs). That is, this is a case where there are three laser spectra in the reflected light spectrum of FBG. As has already been described, since the reflected light spectrum of FBG is not an ideal rectangular wave as illustrated in the figure, the reflected light spectrum calculated by multiplication is similar to sine wave intensity modulated light. According to a sampling theorem, the position information, wavelength information and intensity information can be calculated from the reflected light spectrum. That is, when there are three or more laser spectra in the reflected light spectrum of FBG, the position information and wavelength information can be calculated reliably.

Here, the wavelength resolution (Δλ) can be calculated by the following equation.

ABS(Δλ)=(λ₀²/c)×fs

Here, to obtain three or more laser spectra in the reflected light spectrum of FBG, Δf_{FBG} may be widened or step frequency fs may be increased. To widen Δf_{FBG}, the bandwidth of the FBG section 3, that is, the full width at half maximum of the reflected light spectrum is widened. The bandwidth of the FBG sensor section 3 currently available on the market is, for example, 0.05 nm to 4 nm.

That is, in a publicly known light sensing system using a continuous wavelength sweep laser, the FBG sensor section 3 can more accurately detect reflected light having a narrower full width at half maximum. By contrast, the medical instrument of the present embodiment increases the bandwidth of the FBG sensor section 3 according to the step (λs) of light outputted from the discrete wavelength sweep laser. If (Δf_{FBG}≧2fs), the position information, wavelength information and intensity information may be calculated, but if (Δf_{FBG}≧3fs), the position information, wavelength information and intensity information can be calculated reliably, and (Δf_{FBG}≧4fs) is particularly preferable from the standpoint of accuracy. An upper limit of (Δf_{FBG}) is, for example, on the order of (20×fs) with the above described design bandwidth. When the value exceeds Δfopt in FIG. 13, it is difficult to process reflected light from the optical fiber sensor having many FBG sensor sections 3.

On the other hand, although fs is determined by the specification of the light source 6, fs is 0.1 nm to 0.4 nm with a currently available SSG-DBR laser.

The medical instrument 1 according to the present embodiment using the discrete wavelength sweep laser as the light source determines a measurement range, that is, measurable length from the wavelength resolution (Δλ). Hereinafter, when the insertion portion 12 within a predetermined measurement range is deformed into an arc shape (P0-P1-P2), the measurement accuracy which is an amount of detectable deformation is assumed to be a maximum length d from a chord (P0P2) to an arc of the measurement portion (see FIG. 17). Shape measurement of the insertion portion 12 requires movement on the order of 10 mm to be detected as measurement accuracy d.

With the medical instrument 1 having the light source 6 with 400 channels that outputs light by stepwise changing the light at an wavelength interval (λs) of 0.1 nm in a band of 1533.17 to 1574.13 nm, measurement accuracy of 4.4 mm was confirmed within a measurement range of 0.25 m and measurement accuracy of 8.8 mm was confirmed within a measurement range of 0.5 m.

As described above, since the medical instrument 1 of the present embodiment is a light sensing system using an OFDR scheme, it is possible to reduce the diameter of the optical fiber sensor 2 and using a wideband wavelength variable laser, which is a cheap and discrete wavelength sweep laser, the medical instrument 1 of the present embodiment realizes a diameter reduction as well as a price reduction.

Furthermore, even using a discrete wavelength sweep laser, the medical instrument 1 in which the full width at half maximum Δλ_{FBG} of reflected light from the FBG sensor section 3 is twice or more the predetermined interval λs of the wavelength of light outputted from the light source can calculate the position information and wavelength information.

When the optical fiber sensor 2 is inserted into the insertion portion 12, if the shape of the insertion portion 12 matches the shape of the optical fiber sensor 2 to an extent that would cause no practical problem, the two can be fixed even gently without any problem. The two can be fixed gently by inserting the optical fiber sensor 2 into the channel as described above or the optical fiber sensor 2 may be built in the insertion portion 12 beforehand.

The medical instrument 1 of the present embodiment uses three optical fiber sensors to measure the three-dimensional shape of the insertion portion 12, but any number of optical fiber sensors may be used if it is at least 3. For example, four or more optical fiber sensors may be used to improve measurement accuracy. To measure a wider range, for example, a number of optical fiber sensors which is a multiple of 3 may also be used. That is, using a plurality of sets of optical fiber sensors, each set being composed of three, the region where the FBG sensor section 3 is formed may be shifted and disposed in the longitudinal direction of the insertion portion 12.

### Second Embodiment

Hereinafter, a medical instrument 1B of a light sensing system according to a second embodiment of the present invention will be described with reference to the accompanying drawings. Since the configuration and operations of the medical instrument 1B are similar to those of the medical instrument 1 of the first embodiment, the same components will be assigned the same reference numerals and descriptions thereof will be omitted.

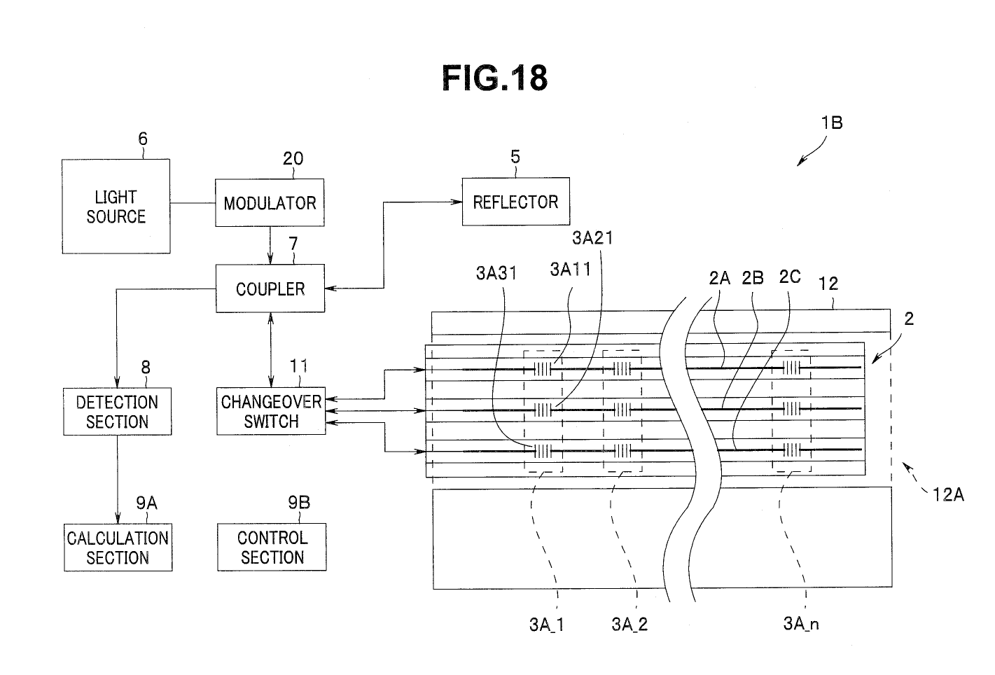

As shown in FIG. 18, the medical instrument 1B includes a modulator 20 which is wavelength modulation means in addition to the configuration of the medical instrument 1 of the first embodiment. The modulator 20 further modulates light beams of wavelengths of a (first) predetermined interval λs which is generated by the light source 6 and stepwise changed into light beams of wavelengths of a second predetermined interval λs2, which is a narrower interval within the predetermined interval λs, and sequentially stepwise outputs the modulated light beams to the coupler 7.

As described with the medical instrument 1 of the first embodiment, it is preferable to increase fs to increase the number of peaks of a spectrum of reflected light of a laser in a reflected light spectrum of FBG. However, according to the current technical standard, it is difficult to realize, for example, 0.04 nm as fs, that is, λs in terms of wavelength even with a light source in the research stage. By contrast, the medical instrument 1B can modulate the second predetermined interval λs2 into less than 0.04 nm through the modulator 20.

As the modulator 20, a phase modulator or acoustic optical device may be used. The phase modulator is a device in which the refractive index of an optical transmission medium varies according to an electric signal inputted, and is, for example, a single crystal Pockels effect device such as lithium niobate (LN). On the other hand, the acoustic optical device is a device that adheres a piezoelectric device to a single crystal acoustic optical medium made of tellurium dioxide (TeO₂), lead molybdate (PbMoO₄) or the like, applies an electric signal to the piezoelectric device to generate ultrasound and causes the ultrasound to propagate in the medium and thereby uses an acoustic optical effect that light passing through the medium is diffracted.

In the medical instrument 1B, when, for example, the wavelength step of light generated by the light source 6, that is, the first predetermined interval λs is 0.1 nm and the measurement range is 2 m, the modulator 20 modulates the second predetermined interval λs2 up to 10 pm and thereby significantly improves the measurement accuracy from 94 mm to 0.6 mm.

That is, the medical instrument 1B of the second embodiment can further improve measurement accuracy in addition to the effects of the medical instrument 1 of the first embodiment.

Although the medical instrument that measures the shape of the insertion portion 12 of the endoscope has been described so far as an embodiment of the light sensing system, the light sensing system of the present invention is not limited thereto, but is also applicable to an industrial endoscope, fatigue analysis for vehicles and buildings, apparatus for measuring characteristics of optical parts, crime prevention system or the like.

As described so far, the present invention is not limited to the aforementioned embodiments, but various modifications, alterations or the like can be made without departing from the spirit and scope of the present invention.

As described so far, the endoscope system of the present embodiment is provided with a light sensing system including three optical fiber sensors disposed in an insertion portion of an endoscope in which a plurality of fiber Bragg grating sensor sections are formed, a light source which is a wideband wavelength variable laser light source that stepwise changes and outputs light beams of wavelengths of a predetermined interval, a wavelength modulation section that modulates the light outputted from the light source into light beams of wavelengths of a second predetermined interval which is a narrower interval within the predetermined interval and stepwise outputs the modulated light beams to a coupler, the coupler that supplies the light outputted from the wavelength modulation section to the optical fiber sensor and also supplies the light to a reflection section, a reflection section that forms reference light to be caused to interfere with the reflected light from the optical fiber sensors, an interference section that generates interference light from the reflected light from the optical fiber sensors and the reference light from the reflection section, a detection section that detects the interference light from the interference section and a calculation section that calculates amounts of deformation of the plurality of fiber Bragg grating sensor sections based on the detection result of the detection section and calculates a three-dimensional shape of the insertion portion.

### (Addendum 1)

A light sensing system including:

an optical fiber sensor in which a plurality of fiber Bragg grating sensor sections are formed;

a light source that outputs light by stepwise changing wavelengths with a passage of time at a wavelength interval of ½ times or less a full width at half maximum of a reflected light spectrum determined by characteristics of the fiber Bragg grating sensor sections;

light supply means for supplying the light outputted from the light source to the optical fiber sensor;

reference light forming means for forming reference light to be caused to interfere with reflected light from the optical fiber sensor from the light outputted from the light source;

interference means for generating the interference light by causing the reference light to interfere with the reflected light;

detection means for detecting the interference light from the interference means; and

a calculation section for calculating amounts of deformation of the plurality of fiber Bragg grating sensor sections based on the detection result of the detection means.

### (Addendum 2)

The light sensing system according to addendum 1, further including wavelength modulation means for modulating the light outputted from the light source into light beams of wavelengths of a second predetermined interval which is a narrower interval within the predetermined interval and stepwise outputting the modulated light beams to the light supply means.

### (Addendum 3)

The light sensing system according to addendum 2, wherein the wavelength modulation means is a phase modulator or acoustic optical device.

### (Addendum 4)

The light sensing system according to addendum 2, wherein the second predetermined interval is less than 0.04 nm and equal to or more than 10 pm.

### (Addendum 5)

An endoscope system comprising a light sensing system, comprising:

an optical fiber sensor disposed in an insertion portion of an endoscope in which a plurality of fiber Bragg grating sensor sections are formed;

a light source that outputs light by stepwise changing wavelengths with a passage of time at a wavelength interval of ½ times or less a full width at half maximum of a reflected light spectrum determined by characteristics of the fiber Bragg grating sensor sections;

light supply means for supplying the light outputted from the light source to the optical fiber sensor;

reference light forming means for forming reference light to be caused to interfere with reflected light from the optical fiber sensor from the light outputted from the light source;

interference means for generating the interference light by causing the reference light to interfere with the reflected light;

detection means for detecting the interference light from the interference means; and

a calculation section for calculating amounts of deformation of the plurality of fiber Bragg grating sensor sections based on the detection result of the detection means.

### (Addendum 6)

The endoscope system according to addendum 5, further including wavelength modulation means for modulating the light outputted from the light source into light beams of wavelengths of a second predetermined interval which is a narrower interval within the predetermined interval and stepwise outputting the modulated light beams to the light supply means.

### (Addendum 7)

The endoscope system according to addendum 6, wherein the second predetermined interval is less than 0.04 nm and equal to or more than 10 pm.

What is claimed is:

1. A light sensing system comprising:
an optical fiber sensor in which a plurality of fiber Bragg grating sensor sections are formed;
a light source that outputs light by stepwise changing frequencies with a passage of time at a frequency interval of ½ times or less a full width at half maximum of a reflected light spectrum determined by characteristics of the fiber Bragg grating sensor sections;
a light supply section that supplies the light outputted from the light source to the optical fiber sensor;
a reference light forming section that forms reference light to be caused to interfere with reflected light from the optical fiber sensor;
an interference section that generates interference light by causing the reference light to interfere with the reflected light;
a detection section that detects the interference light from the interference section; and
a calculation section that calculates amounts of deformation of the plurality of fiber Bragg grating sensor sections based on the detection result of the detection section.

2. The light sensing system according to claim 1, wherein the light supply section is a light splitting section that supplies the light outputted from the light source to the optical fiber sensor and also supplies the light to the reference light forming section, and
the reference light forming section includes a reflection section that causes the interference section to reflect light from the light splitting section as reference light.

3. The light sensing system according to claim 1, wherein the light source is a wideband wavelength variable laser light source.

4. The light sensing system according to claim 1, further comprising three or more of the optical fiber sensors,
wherein the fiber Bragg grating sensor sections are formed at the same position in an axial direction of the three or more optical fiber sensors.

5. The light sensing system according to claim 4, wherein the optical fiber sensors are disposed in an insertion portion of an endoscope system, and
the calculation section measures a three-dimensional shape of the insertion portion.

6. An endoscope system including a light sensing system, the light sensing system comprising:
an optical fiber sensor disposed in an insertion portion of an endoscope in which a plurality of fiber Bragg grating sensor sections are formed;
a light source that outputs light by stepwise changing frequencies with a passage of time at a frequency interval of ½ times or less a full width at half maximum of a reflected light spectrum determined by characteristics of the fiber Bragg grating sensor sections;
a light supply section that supplies the light outputted from the light source to the optical fiber sensor;
a reference light forming section that forms reference light to be caused to interfere with reflected light from the optical fiber sensor;
an interference section that generates interference light by causing the reference light to interfere with the reflected light;
a detection section that detects the interference light from the interference section; and
a calculation section that calculates amounts of deformation of the plurality of fiber Bragg grating sensor sections and the shape of the insertion portion based on the detection result of the detection section.

7. The endoscope system according to claim 6, wherein the light supply section is a light splitting section that supplies the light outputted from the light source to the optical fiber sensor and also supplies the light to the reference light forming section, and
the reference light forming section includes a reflection section that causes the interference section to reflect light from the light splitting section as reference light.

8. A light sensing system comprising:
an optical fiber sensor in which a plurality of fiber Bragg grating sensor sections are formed;
a light source that outputs light by stepwise changing frequencies with a passage of time at a frequency interval of ½ times or less a full width at half maximum of a reflected light spectrum determined by characteristics of the fiber Bragg grating sensor sections;
light supply means for supplying the light outputted from the light source to the optical fiber sensor;
reference light forming means for forming reference light to be caused to interfere with reflected light from the optical fiber sensor;
interference means for generating interference light by causing the reference light to interfere with the reflected light;
detection means for detecting the interference light from the interference means; and
a calculation section that calculates amounts of deformation of the plurality of fiber Bragg grating sensor sections based on the detection result of the detection means.

9. An endoscope system including a light sensing system, the light sensing system comprising:
an optical fiber sensor disposed in an insertion portion of an endoscope in which a plurality of fiber Bragg grating sensor sections are formed;
a light source that outputs light by stepwise changing frequencies with a passage of time at a frequency interval of ½ times or less a full width at half maximum of a reflected light spectrum determined by characteristics of the fiber Bragg grating sensor sections;
light supply means for supplying the light outputted from the light source to the optical fiber sensor;
reference light forming means for forming reference light to be caused to interfere with reflected light from the optical fiber sensor;
interference means for generating interference light by causing the reference light to interfere with the reflected light;
detection means for detecting the interference light from the interference means; and
calculation means for calculating amounts of deformation of the plurality of fiber Bragg grating sensor sections and the shape of the insertion portion based on the detection result of the detection means.
